(19) United States
(12) Patent Application Publication
(10) Pub. No.: US 2016/0279001 A1
(43) Pub. Date: Sep. 29, 2016

(54) FLUID SHIELD ASSEMBLY

(71) Applicant: Brittney Price, South Jordan, UT (US)

(72) Inventor: Brittney Price, South Jordan, UT (US)

(21) Appl. No.: 14/670,467

(22) Filed: Mar. 27, 2015

(51) Int. Cl. A61F 13/84; A61F 13/56

(52) U.S. Cl. CPC A61F 13/84; A61F 13/5622

(57) ABSTRACT

A fluid shield assembly includes a first pad. The first pad is removably coupled to a diaper. The first pad is positioned between the diaper and an article of clothing when the diaper and the article of clothing are worn by a user. The first pad protects the article of clothing from fluid escaping the diaper. A second pad is removably coupled to the article of clothing, providing an additional layer of protection from the fluid escaping diaper.

### BACKGROUND OF THE DISCLOSURE

### Field of the Disclosure

The disclosure relates to shield devices and more particularly pertains to a new shield device for preventing a fluid from escaping a diaper and contaminating an article of clothing.

### SUMMARY OF THE DISCLOSURE

An embodiment of the disclosure meets the needs presented above by generally comprising a first pad. The first pad is removably coupled to a diaper. The first pad is positioned between the diaper and an article of clothing when the diaper and the article of clothing are worn by a user. The first pad protects the article of clothing from fluid escaping the diaper. A second pad is removably coupled to the article of clothing, providing an additional layer of protection from the fluid escaping diaper.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a fluid shield assembly according to an embodiment of the disclosure.

FIG. 2 is a back view of an embodiment of the disclosure.

FIG. 3 is an in-use view of an embodiment of the disclosure.

FIG. 4 is a cross sectional view taken along line 4-4 of FIG. 1 of an embodiment of the disclosure.

FIG. 5 is a front view of an embodiment of the disclosure.

FIG. 6 is a front perspective in-use view of an embodiment of the disclosure.

### DESCRIPTION OF THE PREFERRED EMBODIMENT

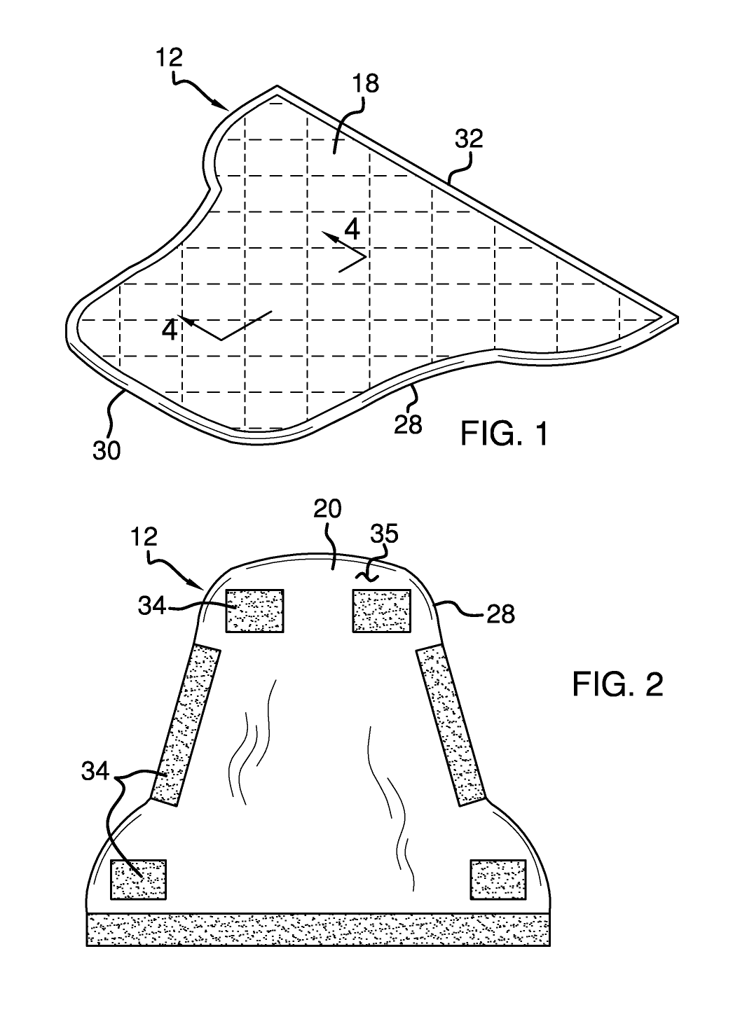

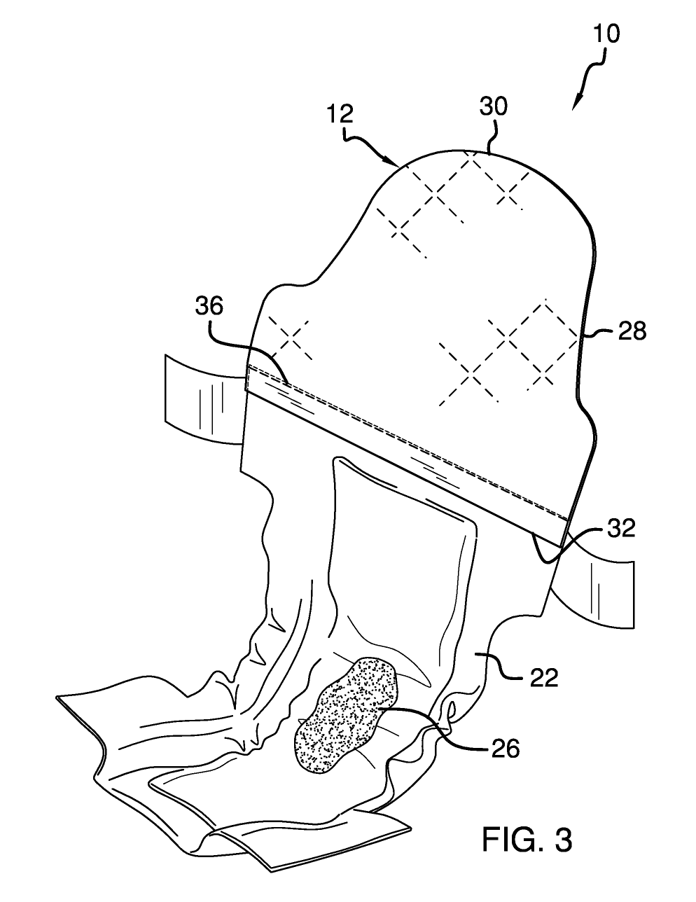

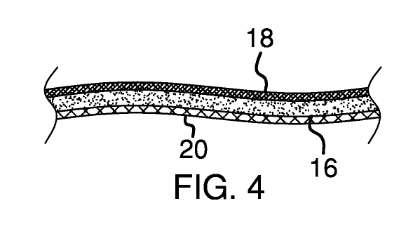

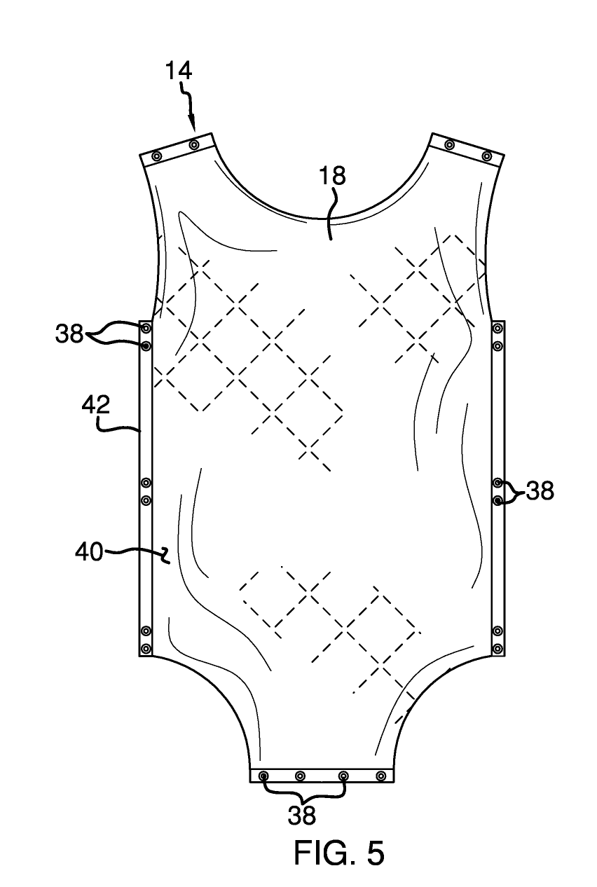

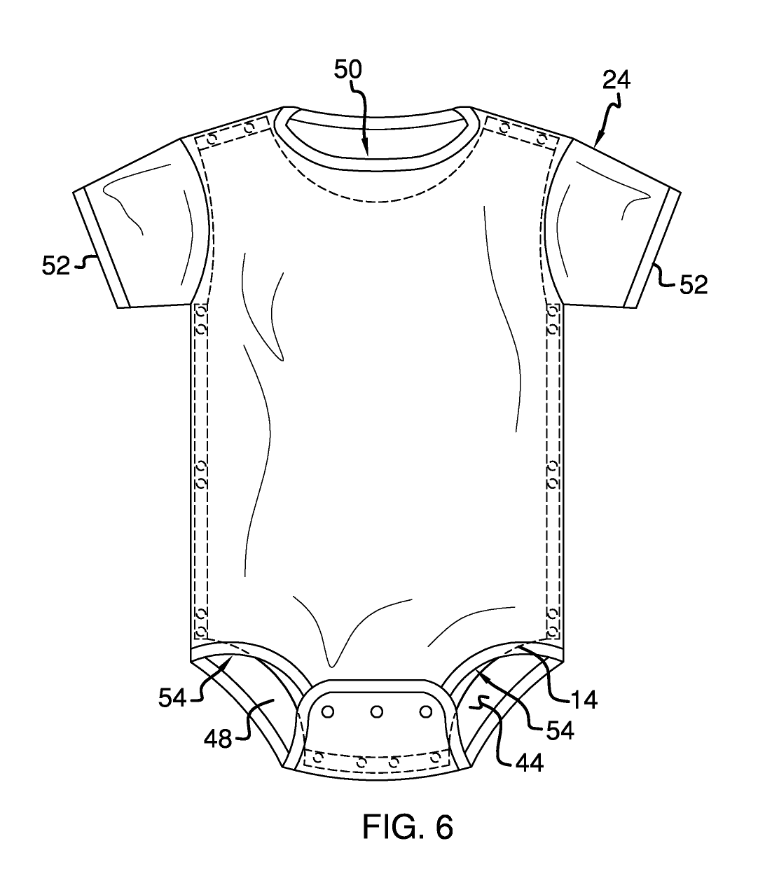

With reference now to the drawings, and in particular to FIGS. 1 through 6 thereof, a new shield device embodying the principles and concepts of an embodiment of the disclosure and generally designated by the reference numeral 10 will be described.

As best illustrated in FIGS. 1 through 6, the fluid shield assembly 10 generally comprises a first pad 12 and a second pad 14. Each of the first pad 12 and the second pad 14 comprise a respective absorbent layer 16 positioned between each of a respective front layer 18 and a respective back layer 20. Each of the front layer 18 and back layer 20 may be comprised of a cloth used in lining the exterior of conventional diapers. The absorbent layer 16 may be comprised of a fluid absorbing material of the type used in conventional diapers.

The first pad 12 is removably coupled to a diaper 22. The first pad 12 is positioned between the diaper 22 and an article of clothing 24 when the diaper 22 and the article of clothing 24 are worn by a user. The first pad 12 protects the article of clothing 24 from a fluid 26 escaping the diaper 22. The fluid 26 may comprise human waste. The diaper 22 may be an infant diaper or the like. The article of clothing 24 may be infant pajamas or the like.

An outer edge 28 of the first pad 12 has a top side 30 that is curved with respect to a bottom side 32 of the outer edge 28, giving the first pad 12 a bell shape. A plurality of couplers 34 is each coupled to an exposed surface 35 of the back layer 20 corresponding to the first pad 12. Each of the couplers 34 is positioned adjacent to and distributed around the outer edge 28. Additionally, each of the couplers 34 may be an adhesive fastener or the like.

The plurality of couplers 34 includes a first coupler 37 positioned adjacent to the bottom side 32 engages the diaper proximate a top edge 36 of the diaper 22. The top side 30 of the outer edge 28 is directed upwardly from the diaper 22. The remaining couplers 34 engage the article of clothing 24, retaining the first pad 12 between the diaper 22 and the article of clothing 24. Additionally, the first pad 12 forms a fluid restricting barrier between the diaper 22 and the article of clothing 24.

The second pad 14 is removably coupled to the article of clothing 24 to provide an additional layer of protection from the fluid 26 escaping the diaper 22. Each of a plurality of fasteners 38 is coupled to an exposed surface 40 of the front layer 18 corresponding to the second pad 14. Each of the fasteners 38 is positioned adjacent to and distributed around an exterior edge 42 of the second pad 14. Each of the fasteners 38 engages an inner surface 44 of the article of clothing 24, retaining the second pad 14 between the article of clothing 24 and the diaper 22.

The second pad 14 is positioned so the back layer 20 corresponding to the second pad 14 abuts the inner surface 44 of a back side 48 of the article of clothing 24. The second pad 14 forms a fluid restricting barrier between the article of clothing 24 and the diaper 22. The exterior edge 42 is shaped such that the second pad 14 is aligned with each of a neck opening 50, each of a pair of arms 52 and each of a pair of leg openings 54 of the article of clothing 24 when the second pad 14 is coupled to the article of clothing 24.

In use, the first pad 12 and the second pad 14 are used in the care of an infant. The first pad 12 and the second pad 14 prevent the article of clothing 24 from becoming soiled by waste from the infant. Each of the first pad 12 and the second pad 14 may be disposed of once the first pad 12 and the second pad 14 become soaked with the fluid 26.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A fluid shield assembly comprising:
a first pad, said first pad being removably coupled to a diaper, said first pad being positioned between the diaper and an article of clothing when the diaper and the article of clothing are worn by a user wherein said first pad protects the article of clothing from fluid escaping the diaper; and
a second pad, said second pad being removably coupled to the article of clothing wherein said second pad provides an additional layer of protection from the fluid escaping diaper.

2. The assembly according to claim 1, wherein each of said first pad and said second pad comprising a respective absorbent layer positioned between each of a respective front layer and a respective back layer.

3. The assembly according to claim 2, further comprising a plurality of couplers, each of said couplers being coupled to an exposed surface of said back layer corresponding to said first pad, each of said couplers being positioned adjacent to and distributed around an outer edge of said first pad, each of said couplers engaging the diaper and the article of clothing such that said first pad is retained between the diaper and the article of clothing, thereby forming a fluid restricting barrier between the diaper and the article of clothing.

4. The assembly according to claim 3, further comprising said outer edge having a top side being curved with respect to a bottom side of said outer edge such that said first pad has a bell shape.

5. The assembly according to claim 2, further comprising a plurality of fasteners each coupled to an exposed surface of said front layer corresponding to said second pad, each of said fasteners being positioned adjacent to and distributed around an exterior edge of said second pad, each of said fasteners engaging an inner surface of the article of clothing such that said second pad is retained between the article of clothing and the diaper, thereby forming a fluid restricting barrier between the article of clothing and the diaper.

6. The assembly according to claim 5, further comprising said exterior edge being shaped such that said second pad is aligned with each of a neck opening, each of a pair of arms and each of a pair of leg openings of the article of clothing when said second pad is coupled to the article of clothing.

7. A fluid shield assembly comprising:
a first pad and a second pad, each of said first pad and said second pad comprising a respective absorbent layer positioned between each of a respective front layer and a respective back layer;
said first pad being removably coupled to a diaper, said first pad being positioned between the diaper and an article of clothing when the diaper and the article of clothing are worn by a user wherein said first pad protects the article of clothing from fluid escaping the diaper, an outer edge of said first pad having a top side being curved with respect to a bottom side of said outer edge such that said first pad has a bell shape;
a plurality of couplers, each of said couplers being coupled to an exposed surface of said back layer corresponding to said first pad, each of said couplers being positioned adjacent to and distributed around said outer edge, each of said couplers engaging the diaper and the article of clothing such that said first pad is retained between the diaper and the article of clothing, thereby forming a fluid restricting barrier between the diaper and the article of clothing;
said second pad being removably coupled to the article of clothing wherein said second pad provides an additional layer of protection from the fluid escaping diaper,
a plurality of fasteners each coupled to an exposed surface of said front layer corresponding to said second pad, each of said fasteners being positioned adjacent to and distributed around an exterior edge of said second pad, each of said fasteners engaging an inner surface of the article of clothing such that said second pad is retained between the article of clothing and the diaper, thereby forming a fluid restricting barrier between the article of clothing and the diaper; and
said exterior edge being shaped such that said second pad is aligned with each of a neck opening, each of a pair of arms and each of a pair of leg openings of the article of clothing when said second pad is coupled to the article of clothing.
